(19) United States
(12) Patent Application Publication
(10) Pub. No.: US 2020/0253193 A1
(43) Pub. Date: Aug. 13, 2020

(54) HEALTHCARE TRANSPORT TOTE AND CONDITION TRACKING THEREOF

(71) Applicant: Intellitote LLC, Germantown, TN (US)

(72) Inventors: Carey Bryant, Hernando, MS (US); Thomas Watts, Round Rock, TX (US); Tom Reed, Incline Village, NV (US); William B. Austin, Germantown, TN (US)

(21) Appl. No.: 16/785,103

(22) Filed: Feb. 7, 2020

Related U.S. Application Data
(60) Provisional application No. 62/802,591, filed on Feb. 7, 2019.

(51) Int. Cl. A01N 1/02; G06Q 10/08; G16H 10/40; G06F 16/27; A61J 1/16; H04Q 9/02; G08B 21/18

(52) U.S. Cl. CPC A01N 1/0242; G06Q 10/0832; G16H 10/40; G06F 16/27; A61J 1/165; H04Q 2209/823; G08B 21/182; A01N 1/0284; A61J 2200/40; A61J 2200/72; H04Q 2209/40; H04Q 9/02

(57) ABSTRACT

An apparatus includes, in one example, a tote configured to maintain a sterilized environment for carrying at least item selected from biological specimens, healthcare-related items, healthcare supplies, etc., an identifier coupled to the tote for tracking the tote, and a sensing module for capturing environmental data related to predetermined risks associated with environmental conditions to which the at least one healthcare-related item is subjected within the tote. A method includes tracking a tote configured to maintain a sterilized environment for carrying at least one healthcare-related item selected from healthcare-related items and healthcare supplies, determining whether the at least one healthcare-related item presents one or more predetermined health-related risks based on environmental data captured for an interior portion of the tote, and communicating a predetermined risk mitigation action in response to determining that at least one healthcare-related item presents the predetermined health-related risks. A system may implement the method.

### CROSS-REFERENCE TO RELATED APPLICATION

This application claims the benefit of U.S. Provisional Patent Application No. 62,802,591, filed Feb. 7, 2019, which is incorporated herein by reference.

### FIELD

This disclosure relates generally to healthcare-related items carriers and, more particularly to a healthcare transport tote and condition tracking thereof.

### BACKGROUND

Healthcare facilities often transport or have third parties transport healthcare items such as biological specimens, healthcare supplies, and/or healthcare-related items. Depending on the transport duration, locations, and environmental conditions of the healthcare items within a tote during transport, healthcare items that have not been properly maintained in accordance with one or more predetermined conditions may present a risk to patients, practitioners, and/or others.

### SUMMARY

The subject matter of the present application provides examples of an apparatus, system, and method for a secure healthcare tote with environmental chain of custody tracking. Accordingly, the subject matter of the present application has been developed in response to the present state of the art and more particularly in response to the shortcomings of conventional healthcare-related items totes with regard to transport tote condition tracking.

Disclosed here is healthcare transport tote and a tracking apparatus for tracking conditions of the tote. The tote is configured to maintain a sealed environment for carrying at least one healthcare-related item selected from biological specimens, healthcare-related items, healthcare supplies, and combinations thereof within. The apparatus further includes an identifier coupled to the tote for tracking the tote and a sensing module having one or more sensors and configured to couple to the tote to capture environmental data related to predetermined risks associated with environmental conditions to which the at least one healthcare-related item is subjected within the tote.

In certain implementations, a condition tracking apparatus is disposed at least partially within the tote and configured to maintain a traceable record of environmental conditions of an interior of the compartment during transportation of the at least one healthcare item within the compartment of the tote. In various implementations, the condition tracking apparatus is configured to communicate distributed ledger transactions comprising environmental data associated with the compartment of the tote during transport.

In various examples, the one or more sensors may include time trackers, temperature sensors, humidity sensors, location sensors, movement sensors, light sensors, pressure sensors, closure status sensors, and so forth.

In some implementations, the sensing module further includes a communication interface configured to communicate environmental data associated with the compartment of the tote during transport. In various examples, the communication interface may be a wireless transmitter, a wireless transceiver, a built-in display, an externally coupled display, and the fourth.

In some examples, the sensing module is configured to be removably disposed on an interior portion of the tote with the sealable closure in a closed disposition throughout transport. In certain implementations, the sealable closure is securely lockable in a closed disposition to prevent physical access to an interior portion of the tote.

In one example, the apparatus further includes a sub-compartment within the tote that is configured to hold an environment regulator selected from a passive temperature regulator, an active temperature regulator, a passive humidity regulator, an active humidity regulator, and so forth. In various examples, the apparatus includes a rechargeable power source with a charging interface configured to enable concurrent leadless charging of one or more instances of the sensing module when removed from an interior portion of one or more totes.

In another example, the charging interface includes stackable charging contacts configured to concurrently electrically couple the one or more instances of the sensing module stacked together with a charger. In some implementations, the charging interface includes a wireless charging interface that inductively couples one or more instances of the sensing module to a wireless charger.

In various implementations, the tote is made of materials configured to substantially retain mechanical integrity and environmental sealability of the tote throughout a plurality of healthcare-related items cleaning processes performed on an interior and an exterior of the tote with the sealable closure in an open disposition. In some implementations, the healthcare-related items cleaning processes are performed at temperature and time parameters that are predetermined to ensure sterilization of an interior and an exterior of the tote. In certain implementations, an exterior surface of the tote includes one or more stacking features that facilitate stacking of two or more instances of a component of the tote selected from the sealable closure separated from the compartment, the compartment separated from the sealable closure, and the sealable closure coupled to the compartment.

Also disclosed herein is a condition monitoring apparatus for monitoring a condition of a tote, wherein the tote is configured to maintain a safe environment for carrying at least one healthcare-related item selected from biological specimens, healthcare-related items, healthcare supplies, and combinations thereof. The apparatus includes a status module that determines whether the at least one healthcare-related item presents one or more predetermined health-related risks based on environmental data captured for an interior portion of the tote, and an action module that communicates a predetermined risk mitigation action in response to determining that at least one healthcare-related item presents the one or more predetermined health-related risks based on one or more values of the environmental data captured being outside a predetermined range.

According to certain implementations, the condition monitoring apparatus includes a location module that tracks the tote using an identifier coupled to the tote.

In certain implementations, the location module, the status module, and the action module are disposed on-board the tote and are configured to determine in real-time whether the predetermined risks are presented without relying on communications with devices external to the tote.

In various implementations the location module tracks the tote using a wireless communications interface that communicates with a location sensor coupled to the tote. In various implementations, the action module communicates the predetermined risk mitigation action to a computing device over a network.

A method for condition tracking of a healthcare-related items tote is disclosed. The method, in one example, includes tracking a tote comprising a compartment and a sealable closure, wherein the tote is configured to maintain a sterilized environment for carrying at least one healthcare-related item selected from biological specimens, healthcare-related items, healthcare supplies, and combinations thereof using an identifier coupled to the tote. The method further includes determining whether the at least one healthcare-related item presents one or more predetermined health-related risks based on environmental data captured for an interior portion of the tote, and communicating a predetermined risk mitigation action in response to determining that at least one healthcare-related item presents the predetermined health-related risks based on one or more values of the environmental data captured being outside a predetermined range.

In some implementations, the method further includes locking the sealable closure to the compartment in a closed disposition to maintain a sealed environment within the interior portion of the tote during transport. In various implementations, the environmental data comprises one or more parameters such as for example, time, temperature, humidity, location, movement, light, pressure, closure status, etc.

The described features, structures, advantages, and/or characteristics of the subject matter of the subject disclosure can be combined in any suitable manner in one or more examples, including examples and/or implementations. In the following description, numerous specific details are provided to impart a thorough understanding of examples of the subject matter of the subject disclosure. One skilled in the relevant art will recognize that the subject matter of the subject disclosure can be practiced without one or more of the specific features, details, components, materials, and/or methods of a particular example, or implementation. In other instances, additional features and advantages can be recognized in certain examples, examples, and/or implementations that can be not presented in all examples, examples, or implementations. Further, in some instances, well-known structures, materials, or operations are not shown or described in detail to avoid obscuring aspects of the subject matter of the subject disclosure. The features and advantages of the subject matter of the subject disclosure will become more fully apparent from the following description and appended claims or can be learned by the practice of the subject matter as set forth hereinafter.

### BRIEF DESCRIPTION OF THE DRAWINGS

In order that the advantages of the invention will be readily understood, a more particular description of the invention briefly described above will be rendered by reference to specific examples that are illustrated in the appended drawings. Understanding that these drawings depict only typical examples of the invention, and are not, therefore, to be considered to be limiting of its scope, the invention will be described and explained with additional specificity and detail through the use of the accompanying drawings, in which:

FIG. 1 is a schematic block diagram of one example of a system for environmental chain of custody tracking of a secure healthcare tote, in accordance with examples of the subject disclosure;

FIG. 2A is a schematic block diagram of another example of an apparatus for transporting healthcare-related items, according to examples of the subject disclosure;

FIG. 2B is a schematic block diagram of an interior view of a closure for the apparatus of FIG. 2A, according to examples of the subject disclosure;

FIG. 3A is a schematic block diagram of one example of an apparatus for transporting healthcare-related items in a closed disposition, according to examples of the subject disclosure;

FIG. 3B is a schematic block diagram of the example of the apparatus of FIG. 3A of the healthcare tote in an open disposition, according to examples of the subject disclosure;

FIG. 4 is a schematic block diagram of one example of a system for condition tracking of a healthcare tote, according to examples of the subject disclosure;

FIG. 5 is a schematic block diagram of an example application of a system for condition tracking of a healthcare tote, according to examples of the subject disclosure;

FIG. 6 is a schematic flowchart diagram of one example of a method for condition tracking of a healthcare tote, according to examples of the subject disclosure; and

FIG. 7 is a schematic flowchart diagram of another example of a method 700 for condition tracking of a healthcare tote, according to examples of the subject disclosure.

### DETAILED DESCRIPTION

Reference throughout this specification to “one example,” “an example,” or similar language means that a particular feature, structure, or characteristic described in connection with the example is included in at least one example of the subject disclosure. Appearances of the phrases “in one example,” “in an example,” and similar language throughout this specification can, but do not necessarily, all refer to the same example. Similarly, the use of the term “implementation” means an implementation having a particular feature, structure, or characteristic described in connection with one or more examples of the subject disclosure, however, absent an express correlation to indicate otherwise, an implementation can be associated with one or more examples.

The terms “including,” “comprising,” “having,” and variations thereof mean “including but not limited to” unless expressly specified otherwise. An enumerated listing of items does not imply that any or all of the items are mutually exclusive and/or mutually inclusive unless expressly specified otherwise. The terms “a,” “an,” and “the” also refer to “one or more” unless expressly specified otherwise.

Furthermore, the described features, advantages, and characteristics of the examples may be combined in any suitable manner. One skilled in the relevant art will recognize that the examples may be practiced without one or more of the specific features or advantages of a particular example. In other instances, additional features and advantages may be recognized in certain examples that may not be present in all examples.

These features and advantages of the examples will become more fully apparent from the following description and appended claims or may be learned by the practice of examples as set forth hereinafter. Certain terms may be used herein such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive unless expressly specified otherwise. The terms “a,” “an,” and “the” also refer to “one or more” unless expressly specified otherwise.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As will be appreciated by one skilled in the art, aspects of the present invention may be embodied as a system, method, and/or computer program product. Accordingly, aspects of the present invention may take the form of an entirely hardware example, an entirely software example (including firmware, resident software, microcode, etc.) or an example combining software and hardware aspects that may all generally be referred to herein as a “circuit,” “module,” or “system.” Furthermore, aspects of the present invention may take the form of a computer program product embodied in one or more computer readable mediums having program code embodied thereon.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field-programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine-dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to examples of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatuses or other devices to produce a computer implemented process, such that the instructions which execute on the computer, other programmable apparatus, or other device implement the functions/acts specified in the flowchart and/or block diagram block or blocks.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field-programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods, and computer program products according to various examples of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding examples. Indeed, some arrows or other connectors may be used to indicate only the logical flow of the depicted example. For instance, an arrow may indicate a waiting or monitoring period of unspecified duration between enumerated steps of the depicted example. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

According to one example, described herein is a tote with sensors (with wired and/or wireless connections) embedded into the inside of the tote to monitor the environment and provide a documented database of the conditions through the entire shipment process, with optional attachable electronic documentation, with a display mechanism to indicate current internal environment conditions and notify if predetermined environmental conditions are exceeded or are on track to be exceeded.

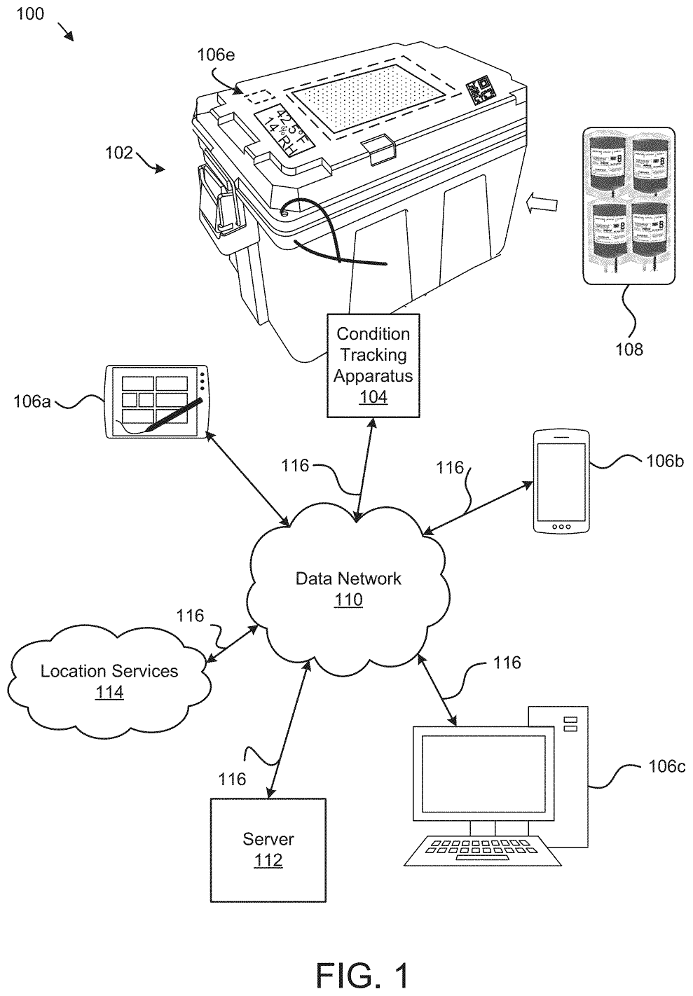

FIG. 1 is a schematic block diagram of one example of a system 100 for condition tracking of a healthcare transport tote 102. In various examples, the tote 102 is configured to transport healthcare items 108, such as biological specimens, healthcare-related items, healthcare supplies, and the like, between healthcare facilities or between locations within a healthcare facility. Various such healthcare items should be maintained within predetermined ranges of environmental parameters during transport. Biological specimens such as blood, plasma, organs, and so forth may need to maintained below a certain temperature during transport. Surgical instruments that have been used in a procedure may need to be transported in a sealed environment at a specified temperature to minimize the risk of exposing persons or articles to pathogens or other biohazards.

However, for various reasons, such as negligence, inattention, or workload, healthcare transport totes used to transport such healthcare items may not be immediately sanitized after a prior procedure or before a current procedure, and the sanitation status of the equipment tote may not be easily determined.

Moreover, some healthcare-related items may become contaminated by exposure to environmental influences. For example, some tools or specimens risk contamination if they are exposed to moisture or heat. Additionally, healthcare-related items may be sanitized and subsequently carried (e.g., transported or stored) in a tote before a procedure but the practitioner preparing for or perform the procedure is unaware of whether the healthcare-related items has been exposed to any environmental contamination risks while being carried. Because the condition history of the healthcare-related items may not be clear, practitioners might inadvertently use biological specimens and/or healthcare-related items that has not been properly maintained during transport or that has been exposed to environmental contamination risks, resulting in risks, or practitioners may re-sterilize already sterilized equipment, resulting in a waste of time and resources.

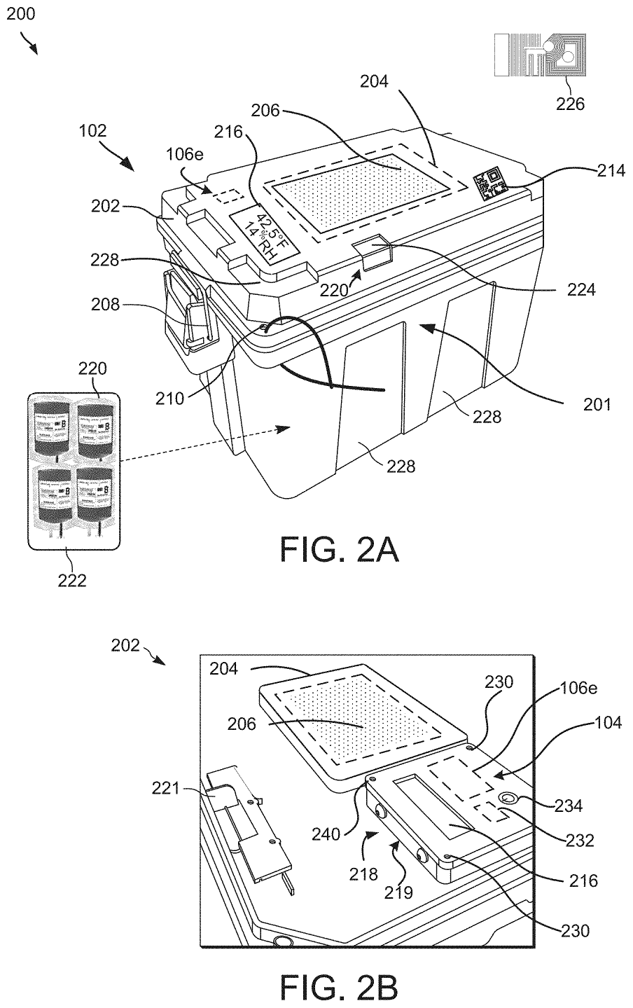

In one example, the system 100 includes a tote 102 configured to maintain a sealed environment for transporting healthcare items such as biological specimens, healthcare-related items, healthcare supplies, and so forth. In various implementations, and the tote 102 includes an identifier for tracking the tote 102 and one or more environmental sensors coupled to the tote and configured to capture environmental data related to predetermined contamination risks for the healthcare-related items carried by the tote 102. The tote 102 is discussed in further detail with respect to the apparatus 200 of FIG. 2A and FIG. 2B.

In various examples, the system 100 includes a condition tracking apparatus 104 with a location module that tracks a location of the tote 102. The tote 102 has a compartment and a sealable closure configured to maintain a sealed environment for carrying within at least one healthcare-related item selected from biological specimens, healthcare-related items, and healthcare supplies. The location module uses an identifier coupled to the tote for tracking the tote and/or conditions within the tote.

The condition tracking apparatus 104 includes a status module that determines whether the healthcare item or items carried within the tote have been exposed to predetermined risks (e.g., temperature or humidity outside predetermined range, duration of transport to long, tote closure opened during transport, and so forth) based on environmental data captured by one or more sensors coupled to the tote.

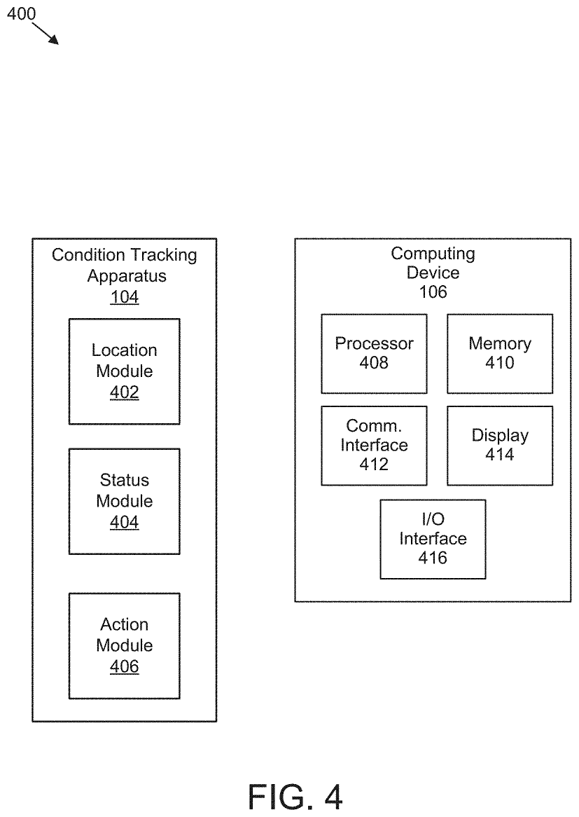

The condition tracking apparatus 104 also include an action module that communicates a predetermined risk mitigation action in response to determining that the tote has been exposed to the predetermined risks based on one or more items of the environmental data captured being outside the predetermined range. The condition tracking apparatus 104 is described in more detail with regard to the apparatus 400 depicted in FIG. 4.

In one example, the system 100 also includes one or more computing devices 106. In various implementation, an embedded computing device 106e is disposed within a portion of the tote 102. In certain implementations, the one or more computing devices 106 include an external computing device, such as for example, a tablet 106a, a smartphone 106b, and/or a personal computer 106c. In certain examples, the condition tracking apparatus 104 is implemented using the embedded computing device 106e within the tote 102 or coupled to the tote 102 and is configured to communicate environmental data captured by the sensors as well as status determinations and recommended actions made using the embedded computing device 106e within the tote 102 to the one or more computing devices 106, that are external to the tote, such as for example, the tablet 106a, the smartphone 106b, and/or the personal computer 106c. More details regarding the one or more computing devices 106 is provided below with respect to apparatus 400 depicted in FIG. 4.

One benefit of implementing the condition tracking apparatus 104 using the embedded computing device 106e within the tote 102 is that in such implementations, the condition tracking apparatus 104 monitors in real-time whether the conditions of the tote have remained within predetermined ranges throughout transport without having to rely on communication with a centralized server during transport. Another benefit of implementing the condition tracking apparatus 104 within the embedded computing device 106d of the tote 102 is that it provides a co-located record establishing a chain of custody for the tote, the contents of the tote, and the environmental data relating to the condition of the tote.

In certain examples, the one or more computing devices 106 communicate over data network 110 with one or more servers 112 and one or more location services 114. For example, in some examples, the condition tracking apparatus 104 within the tote 102 communicates a record establishing a location and condition of the tote during transport over a data network 110 to a server 112 so that the data within the record is recorded as a transaction within a distributed ledger of a blockchain platform or other distributed ledger platform. By communicating condition tracking information as a transaction within a distributed ledger, the information within the record is securely and accurately stored using cryptography to become part of a ledger provides an auditable and immutable history of all transactions. This improves healthcare transport condition tracking by ensuring that in the case of a serious risk materializing, the nature and source of the risk can be confidently identified with a very low likelihood that important condition tracking information will be lost and or tampered with.

In some examples, the data network 110 includes one or more ad hoc networks using one or more wireless connections 116 established directly between the computing devices 106 and the tote 102. The one or more wireless connections 116 may vary from device to device and from application to application. Reference to “the wireless connection” as used herein is merely exemplary and, does not imply that all of the one or more wireless connections 116 are implemented using the same technology or standards. For example, in certain examples, the wireless connection 116 may employ a Radio Frequency Identification (RFID) communication including RFID and/or near field communications (NFC) standards established by the International Organization for Standardization (ISO), the International Electrotechnical Commission (IEC) such as for example ISO/IEC 15443. In some examples, the RFID/NFC standards may also include communication standards established under The American Society for Testing and Materials® (ASTM®), the DASH7™ Alliance, and EPCGlobal™.

In various examples, the wireless connection 116 may also employ a Wi-Fi network based on any one of the Institute of Electrical and Electronics Engineers (IEEE) 802.11 standards. Alternatively, the wireless connection 116 may be a BLUETOOTH® connection.

In certain examples, the wireless connection 116 may employ a ZigBee® connection based on the IEEE 802 standard. In one example, the wireless connection 116 employs a Z-Wave® connection as designed by Sigma Designs®. In other examples, the wireless connection may employ an ANT® and/or ANT+® connection as defined by Dynastream® Innovations Inc. of Cochrane, Canada.

The wireless connection 116 may be an infrared connection including connections conforming at least to the Infrared Physical Layer Specification (IrPHY) as defined by the Infrared Data Association® (IrDA®). All standards and/or connection types include the latest version and revision of the standard and/or connection type as of the filing date of this application. A person of ordinary skill in the art will recognize that although many of the connections depicted herein are beneficially wireless connections, reference to the wireless connection 116 may also refer to a wired connection in situations where a wired connection is suitable. For example, in certain examples, the computer 106c, the server 112, the location services 114, and/or other devices or services may connect to the tracking apparatus 104 through wired connections and/or wireless connections 116.

As depicted in FIG. 4, the one or more computing devices 106, in one example, include one or more processors 408, where the processor 408 may be a central processing unit (“CPU”), a multicore processor, and the like. In various examples, the one or more computing devices 106 may include memory 410 used by the processor 408 and/or for data storage. The one or more computing devices 106 include in some implementations a communications interface 412, a display 414, an I/O interface 416, buses, and other typical computer processing components. In various examples, the one or more computing devices 106 may be embedded within the tote 102 such as the embedded computing device 106e. In some examples, the one or more computing devices 106 may include a desktop computer, a laptop computer, a tablet computer, a smartphone, a pager, a smartwatch, smart assistant and the like.

Many healthcare facilities have a designated area for equipment sterilization that is separate from any treatment room. Such separation may be beneficial because cleaning, sterilizing and storing equipment in the same room where the delivery of patient care is provided may the risk of contamination. Where a healthcare transportation tote is used to carry biological specimens or potentially contaminated healthcare-related items such as surgical tools, a healthcare facility may require the transportation tote itself to be subjected to appropriate cleaning and/or sterilization processes so as to minimize risks to healthcare practitioners and/or patients.

Some existing apparatuses and methods for transporting healthcare-related items to the designated area for equipment sterilization depend on ad hoc or documented procedures which may or may not be followed and which fail to track the location, environmental conditions, and sanitary status of healthcare-related items being transported for sterilization after use and/or being transported after sterilization for use in a further procedure.

The systems, apparatuses, and methods described herein improve healthcare-related items transport technology by combining both mechanical and electronic structures for performing functions to ensure that a healthcare-related items “chain of custody” solution tracks and traces healthcare-related items and the environmental conditions to which the healthcare-related items is subjected to during transport and/or temporary storage to minimize risk of contaminated equipment being used.

FIG. 2A is a schematic block diagram of another example of an apparatus 200 for transporting healthcare-related items, according to one or more examples of the subject disclosure. FIG. 2B is a schematic block diagram of an interior view (e.g., an underside view) of the sealable closure 202 for the apparatus 200 depicted above with respect to FIG. 2A, according to one or more examples of the subject disclosure.

In one example, the apparatus 200 includes one instance of a tote 102 comprising a compartment 201 and a sealable closure 202. In some examples, the compartment 201 and the sealable closure 202 are made of temperature insulating and moisture resistant materials, so that with the sealable closure 202 in a closed disposition, the tote is configured to maintain environmental conditions of an interior of the compartment 201 within predetermined ranges during transportation of healthcare-related items within the compartment 201 of the tote 102. In various implementations, the compartment 201 and the sealable closure 202 are configured to prevent any fluid within the compartment 201 from leaking out of the tote 102. For example, a bottom interior surface of the compartment 201 may include a tray, a depressed shape, an absorbent pad, or similar feature for preventing leaks.

In various examples, the tote 102 is made of materials configured to substantially retain mechanical integrity and environmental sealability of the tote throughout a plurality of healthcare-related items cleaning and/or sterilization processes performed on an interior and an exterior of the tote 102 with the sealable closure 202, 202 in an open disposition.

For example, as described above, a lightweight rigid insulating material such as expanded polypropylene foam or expanded polystyrene foam may be used to minimize the weight of the tote 102 while at the same time providing a predetermined level of insulation against temperature changes and to maintain mechanical integrity environmental sealability even after exposure to multiple cycles of healthcare-related items cleaning processes, such as for example, steam sterilization.

In various examples, the healthcare-related items cleaning processes are performed at temperature and time parameters that are predetermined to ensure sterilization of an interior and an exterior of the tote. For example, the United States Centers for Disease Control and Prevention publishes guidelines for disinfection and sterilization in healthcare facilities. To ensure that possible microorganisms are killed by the cleaning process, steam sterilization performed according to the published guidelines must be performed at a sufficiently high temperature such as for example 121° C. and must be maintained at a specific temperature for a predetermined minimum time to kill a particular type of microorganism. Other standards may be determined by other government authorities, or according to facility guidelines. Other steam cleaning processes may include the dryness of the steam and pressure within the steam sterilization equipment. Thus, certain implementations of the present disclosure are made of materials designed to effectively withstand such conditions without damage or deterioration. Other suitable materials may be selected by those of ordinary skill in the art.

In some examples, a seal (not shown) is disposed between the compartment 201 and the sealable closure 202 and is coupled to either the rim of the compartment 201 or the corresponding rim of the sealable closure 202 the seal may be made of elastomeric material and may be sufficiently thick so as to provide cushioning and/or insulating properties to the tote 102 with the sealable closure 202 in a closed disposition.

In certain examples, the tote 102 includes one or more sub-compartments 204, such as a pouch, a pocket, a notch, or similar enclosure or partial enclosure that is configured to hold one or more environment regulating items 206 used for regulating an environmental condition within the compartment 201. For example, when the tote is being used to transport vaccines, one type of useful environment regulating item 206 may be a gel refrigerant pack or a quantity of dry ice. In other examples, the environment regulating items 206 may be a desiccant pack used to regulate humidity. A further example of an environment regulating item 206 may be an amount of activated charcoal for adsorbing odors, vapors, and so forth. In various implementations, the sub-compartments 204 may be flexible like a pouch or in further implementations the sub-compartments 204 may be rigid like a box or a notch. In some examples, the sub-compartments 204 may be configured to hold an active cooling or heating device such as a Peltier cooling device, a fan, a humidifier, or a resistive heating device.

In various examples, an identifier 214 such as a QR code, barcode, number, text, may be coupled to the tote 102. The identifier 214 may also be digitally stored within the condition tracking apparatus 104 along with condition tracking records for a particular tote 102. Coupling an externally readable identifier 214 to the tote 102 that matches an internally stored copy of the identifier 214 provides a level of redundancy and convenience for ensuring that the tote 102 is properly identified both within the stored data of the condition tracking apparatus 104 and within external systems.

As described above with respect to apparatus 200, the apparatus 200 may also include an RFID/NFC device 226 disposed with or separately from the identifier 214. In certain implementations, the identifier 214 may be disposed on an exterior surface of the tote 102 such as for example on a top portion or on a side portion of the sealable closure 202. In other implementations, the identifier 214 may be an electronically readable identifier within a sensor or the sensing module 219 that wirelessly communicates with a reader used to identify the tote 102.

In some examples, the tote 102, including the compartment 201 and the sealable closure 202 may include stacking features 228 such as recessed areas at the corners on top of the sealable closure 202 or extending from the bottom of a side portion of the compartment 201. The various stacking features 228 may facilitate stacking of two or more instances of a component of the tote 102 such as for example the sealable closure 202 separated from the compartment 201, the compartment 01 separated from the sealable closure. With the complement separated, multiple sealable closures 202 may be stacked together to conserve space occupied when storing or shipping the container. Similarly, multiple compartments 201 may be stacked together to conserve space.

In certain examples, the sealable closure 202 of the tote may be latched or otherwise coupled to the compartment 201 using latches 208. In FIG. 2A, only one latch 208 is depicted at one end of the tote 102. However, any number of latches may be included at desired positions to securely latch the sealable closure 202 to the compartment 201. Furthermore, in some examples, in a closed position, the sealable closure 202 may be locked to the compartment 201 with using a locking feature 210 such as for example, a hole through which a shackle of a padlock or a nylon zip tie may be inserted. In various examples, locking the sealable closure 202 to the compartment 201 prevents accidental and/or intentional unauthorized access to an interior portion of the tote 102.

In some examples, instead of and/or in addition to, a padlock or a nylon zip tie, a tamper-evident seal 212 may be inserted through the locking feature 210 so that instead of having to open a lock or cut a zip tie, which may take time, to access an interior portion of the tote 102, an authorized user may easily break the tamper-evident seal 212 for legitimate purposes such as to remove the needed healthcare-related items and/or healthcare supplies. The healthcare-related items 222 may include sterilizable items such as surgical equipment and in some examples, the healthcare-related items 222 may be loaded to or from the tote 102 using a tray 220.

In various examples, the healthcare supplies (not shown) may include biomedical such as for example blood, plasma, biological specimens, organs, and so forth. In other examples, the healthcare supplies may include pharmaceutical supplies, cooling packs, or similar objects. In addition to preventing contamination based on inadvertent or intentional access to healthcare-related items and/or healthcare supplies, the locking feature 210 and/or the tamper-evident seal 212 may prevent and/or alert against tampering, stealing, or accidentally damaging the healthcare-related items 222 and/or healthcare supplies as a result of opening the sealable closure 202 at a time when it should not be opened, such as for example, during transport.

If the tamper-evident seal 212 is broken, as depicted for example in FIG. 2B, its broken state provides an immediately recognizable mechanical alert signal that the sealable closure 202 of the tote has been opened and thus the healthcare-related items and/or healthcare supplies inside may have been exposed to airborne contaminants or contact with contaminated objects or persons.

Another example of a safety mechanism that facilitates fast and clear handling of the tote 102 by authorized healthcare personnel may include a sanitary state indicator 224 with a first half having a visual indicator such as a green-colored surface to indicate that the tote 102 is currently considered safe to use and/or to access under authorized conditions. A second half of the sanitary state indicator 224 may include a different visual indicator such as a red-colored surface to indicate that the tote 102 is currently considered unsafe to use and/or to access, or is in a contaminated condition and needs to be sterilized using healthcare sterilization equipment at a particular venue.

In some examples, the state of the sanitary state indicator 224 may be seen from a top or a side perspective, as depicted in FIG. 2A. Furthermore, in various implementations, the sanitary state indicator 224 is covered with a clear window to prevent accidental and/or intentional unauthorized changing of the state, e.g. contaminated or sanitary from outside the tote 102. A sliding tab 221 or similar acutator of the sanitary state indicator 224 is in certain implementations configured to be actuated only when the sealable closure 202 is not locked or otherwise secured to the compartment 201. This configuration functions as a safety feature to prevent tampering and/or accidental changing of the sanitary state indicator 224.

In certain examples, the apparatus 200 includes a sensing module 219 that includes one or more sensors and is configured to couple to an interior of the tote 102, such as for example, the underside or inside of the sealable closure 202. Various types of sensors may be included such as time trackers, temperature sensors, liquid sensors, humidity sensors, location sensors, movement sensors such as accelerometers, light sensors, pressure sensors, closure status sensors, etc. For example, in certain implementations, the sensing module 219 includes one or more liquid sensors disposed at a bottom interior surface of the tote 102 to facilitate detection of a fluid that has leaked from a specimen or from a coolant.

In one example, the sensing module 219 includes one or more displays 216 configured to display one or more environmental data parameters sensed by the one or more sensors of the sensing module 219.

In certain examples, the condition tracking apparatus 104 is co-located with the sensing module 219 and may share components such as a processor, memory, power source, and so forth.

In some examples, the sensing module 219 includes a wireless communication interface 232 such as Bluetooth, Wi-Fi, or similar technologies as described above for communicating the environmental data to an external computing device and/or an external display. Thus, the wireless communication interface 232 beneficially enables access to the sensing information even in implementations where the display 216 is not visible from outside the tote 102, for example by transporting personnel. In various examples, the wireless communication interface 232 communicates the data such as tote environment data to remote locations throughout the transport process in real-time.

In various examples, the sensing module 219 is configured to be removably disposed on an interior portion of the tote 102 such as for example at the underside, or inside of the sealable closure 202. In some examples, the sensing module 219 includes a power switch 234 such as a pushbutton, capacitive touch switch, or other switch types suitable for use in equipment may be exposed to biological contaminants and/or sterilization procedures. In some examples, the sensing module includes insulating and water-resistant gaskets and/or other features that facilitate sterilization of the sensing module 219 within or separated from the sealable closure 202.

In one implementation, the sensing module 219 includes a rechargeable power source such as for example a battery pack. In various implementations, the sensing module includes a charging interface configured to enable concurrent leadless charging of one or more instances of the sensing module 219 when removed from an interior portion of one or more totes 102. In some examples, the leadless charging is accomplished through stackable charging contacts 230 configured to concurrently electrically couple one or more instances of the sensing module 219 stacked together with a charger.

Beneficially, using cordless charging reduces the risk of contamination by facilitating a sensing module design without interface access portals and also facilitates efficient and easy charging of the sensing module 219. In other examples, the sensing module 219 includes a wireless charging interface that inductively couples one or more instances of the sensing module 219 to a wireless charger. The sensing module 219 with inductively coupled chargers also beneficially facilitate a module design that minimizes access ports, crevices, cavities and so forth and makes sterilization of the sensing module 219 separated from sealable closure 202, or in other implementations, the sensing module 219 is removably coupled to an interior portion of the compartment 201 and function similarly.

Removably disposing the sensing module 219 at an interior portion of the tote 102 significantly improves condition tracking of the tote 102 as compared to existing technologies where sensors since environmental conditions of a delivery vehicle, warehouse, or other environments external to the interior portion of the tote 102. For example, the tote 102 may be used to transport healthcare supplies that require a temperature significantly lower than an ambient temperature within a delivery vehicle, loading dock, or other location. Existing sensors disposed outside the tote 102 may record a temperature that would be detrimental to the healthcare-related items, and/or supplies within the tote 102.

In some implementations, the tote 102 includes a sub-compartment within the tote that is configured to hold an environment regulator selected from a passive temperature regulator, an active temperature regulator, a passive humidity regulator, an active humidity regulator, and so forth. In various examples, the apparatus includes a rechargeable power source with a charging interface configured to enable concurrent leadless charging of one or more instances of the sensing module when removed from an interior portion of one or more totes.

In certain examples, the apparatuses disclosed herein such as apparatus 200 may be well-insulated and/or may include cooling features within their interior such as for example ice packs, Peltier coolers, dry ice, liquid nitrogen, etc. Therefore, including the one or more sensors of the sensing module 219 within an interior portion of the tote 102 provides a more accurate reading of the environmental conditions within the interior portion of the tote 102.

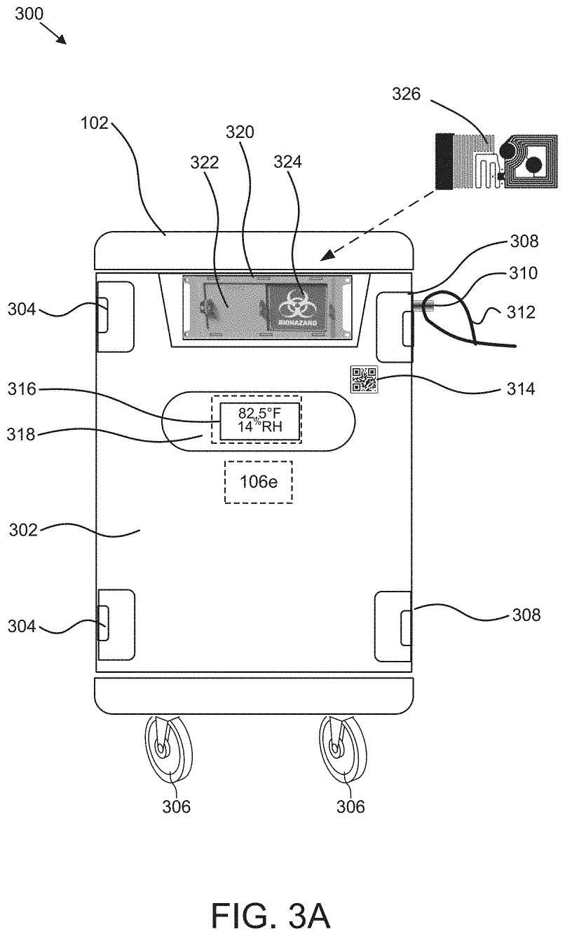

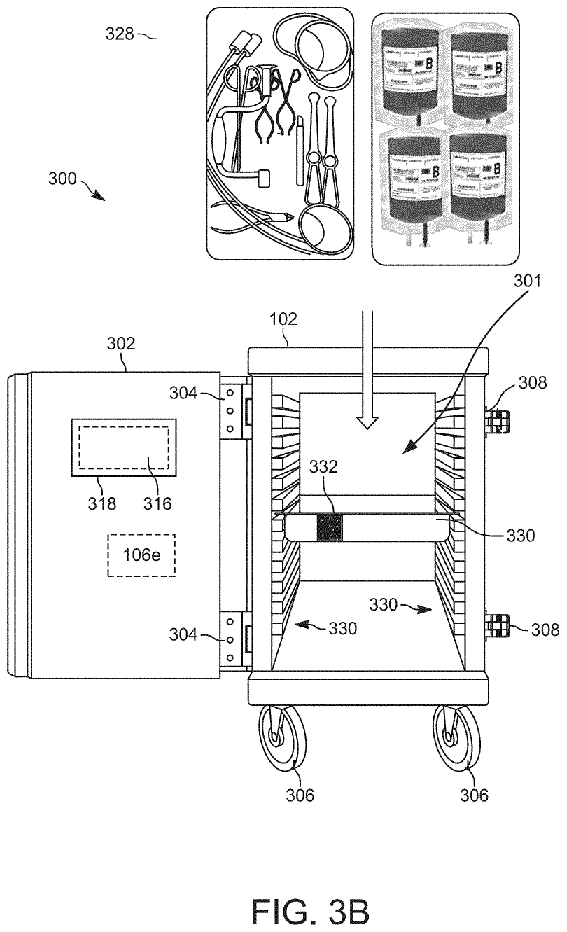

Referring now to FIG. 3A and FIG. 3B, FIG. 3A is schematic block diagram of one example of an apparatus for transporting healthcare-related items in a closed disposition for use with an condition tracking apparatus and/or system and FIG. 3B is schematic block diagram of the example of the apparatus of FIG. 3A of the tote in an open disposition.

In one example, the apparatus 300 includes an instance of a tote 102 that includes a compartment 301 and a sealable closure 302 that may be disposed in a closed or an open disposition to allow access to the interior of the tote 102. For example, in various examples, the sealable closure 302 may be a door, a lid, a cover, an opening, a gate, a barrier, a seal, and the like. In certain examples, the tote 102 may include insulating material that helps maintain an interior the tote 102 at a predetermined temperature. For example, in some examples, a lightweight rigid insulating material such as expanded polypropylene foam or expanded polystyrene foam may be used to minimize the weight of the tote 102 while at the same time providing a predetermined level of insulation against temperature changes. In some examples, the tote 102 may include a lining or coating of materials, such as for example medical grade stainless steel or polymers that heat resistant, moisture resistant, and generally facilitate sterilization of the healthcare-related items being transported as well as the tote 102.

In some examples, the sealable closure 302 of the tote 102 is attached at one side of an opening of the tote 102 by hinges 304. In such examples, the sealable closure 302 may also be latched at an opposing side to the hinges 304 using the latches 308 such as depicted in FIG. 2A. In certain examples, one or more of the latches 308 may include a hasp or similar structure. In various implementations, the compartment 301 and the sealable closure 302 are configured to prevent any fluid within the compartment 301 from leaking out of the tote 102. For example, a bottom interior surface of the compartment 301 may include a tray, a depressed shape, an absorbent pad, or similar feature for preventing leaks.

In various examples, a tamper-evident seal 312 may be attached through or around the latches 308 immediately after closing the sealable closure 302 with healthcare-related items 328 (such as depicted in FIG. 2B) inside the tote 102. In various examples, the sealable closure 302 may include a closure status sensor such as an electromechanical switch or a magnetic switch that is triggered upon opening and/or closing the sealable closure 302 of the tote 102.

In one example, an identifier 314 is coupled to the tote 102. In various examples, the identifier 314 is be used for tracking a location of the tote 102. In some examples, the identifier may be recognized optically such as for example a barcode or 2D barcode that can be recognized by a camera in a computing device 106, such as the tablet 106a or the smartphone 106b.

In various examples, the identifier 314 comprises a wireless identification device such as an RFID/NFC device 326. The identifier 314 may include both a RFID device and an optically readable barcode to provide flexible compatibility with various types of healthcare information systems input devices such as barcode scanners, RFID/NFC readers, and/or portable computing devices e.g. 106a, 106b which may include applications for scanning barcodes using a camera and/or RFID/NFC transceivers.

In various examples, the RFID/NFC devices 326 are compliant with ISO/IEC standard 15443 and data may be read from or written to the RFID/NFC devices 326. In some examples, the identifier 314 is used to determine a location by utilizing location services 114 that determine the location of the computing device 106 based on various location technologies such as global positioning system (GPS), Internet access point address, or a user-programmable location that may be used to distinguish for example different rooms within a building and/or different vehicles. In other words, the location services 114 are not limited to conventional GPS location services but may include user-programmable location identifiers that help establish a chain of custody for the tote 102 during transport.

In other examples, the computing device 106 writes location data to a memory device of the identifier using write functions for the RFID/NFC device 326 rather than merely reading the identifier 314 and mapping the identifier to a certain location based on the time the identifier was read and the location of the device reading the identifier,

In some examples, the apparatus 300 includes a status indicator 320 with a sliding panel 322 that slides to one side to expose a visual indicator 324 such as a colored surface, symbol, word, icon, or another visual indicator that indicates that the healthcare-related items 328 and/or the tote 102 is at risk e.g., out of a predetermined environmental range or contaminated. In such examples, the status indicator slides to the opposite side to indicate that the healthcare-related items 328 and/or the tote 102 is clean e.g. sanitized. In some examples, in addition to the electronic and/or computing elements described herein, the status indicator 320 may include mechanical and/or visual elements similar to those described in U.S. patent application Ser. No. 15/297,036 which is incorporated herein by reference.

In one example, the RFID/NFC device 326 is attached to the backside of the status indicator 320 to facilitate the installation or coupling of both the identifier 314 using the RFID/NFC device 326 and the status indicator 320 together. Similarly, a barcode and/or RFID/NFC device may be installed in some examples on a front portion of the sliding panel 322 of the status indicator 320. In certain examples, the apparatus 300 includes one or more sensors 318 coupled to the tote 102 for capturing environmental data related to potential contamination of the healthcare-related items transported by the tote. For example, temperature and/or humidity outside of predetermined ranges can be considered contamination risks related to the growth of certain types of contaminants including bacteria, viruses, molds, fungi, etc.

In certain examples, the sensors 318 may include temperature sensors, humidity sensors, light sensors, motion sensors, air movement sensors, or any type of sensors configurable to sense a condition related to a predetermined contamination risk around, on, or within the tote 102. For example, in one example, the sensors 318 include a temperature sensor that measures the interior and/or exterior temperature of the tote 102. In some examples, the sensors 318 include a humidity sensor that measures the interior and/or exterior relative humidity of the tote 102. In various examples, the sensors 318 include a wireless transceiver that transmits the environmental data to one or more of the computing devices 106 over the wireless connections 116. In certain examples, the sensors 318 include a display 316 that displays values of the data captured by the sensors 318.

In various examples, the identifier 314 (e.g. the RFID/NFC device), the sensors 318, and/or the status indicator 320, or portions thereof, are designed to withstand predetermined sterilization temperatures and procedures so that the identifier 314, the sensors 318, and/or the status indicator 320, can be sterilized while being coupled to the tote 102. In other examples, the identifier 314, the sensors 318, and/or the status indicator 320 are designed to be detachably coupled to the tote 102 so that they can be sterilized separately from the tote 102.

In further examples, the identifier 314, the sensors 318, and/or the status indicator 320, or portions thereof, are designed to be detachably coupled so that they can be disposed as single-use consumables thus eliminating the need for sterilization. Surprisingly, disposable components such as identifiers 314 (e.g. RFID/NFC devices), sensors 318, and/or status indicator 320 may be more cost-effective than reusable identifiers 314 (e.g. RFID/NFC devices), sensors 318, and/or status indicator 320 because the disposable identifiers 314 (e.g. RFID/NFC devices), sensors 318, and/or status indicator 320 can be designed using low-cost materials and/or manufacturing methods and the costs of failing to detect or avoid preventable contamination risks that result in negative patient outcomes can be extremely high.

As illustrated in FIG. 2B, in some examples, the tote 102 is configured to adjustably accommodate one or more procedure trays 330 that hold healthcare-related items 328 (e.g. surgical, medical, dental, or laboratory instruments and containers). For example, in certain examples, the tote 102 has a number of slots at predetermined intervals to accommodate different heights of procedure trays 330. In various examples, the procedure tray 330 includes a tray identifier 332. The tray identifier 332 in certain examples include an RFID/NFC device and/or an optically readable barcode. The tray identifier 332 can be used to identify one or more specific trays associated with particular procedures.

FIG. 4 is a schematic block diagram of one example of an apparatus 400 for condition tracking of a healthcare-related items tote. In one example, the apparatus 400 includes one instance of the condition tracking apparatus 104 that includes a location module 402 for tracking a location of a tote. In various examples, the apparatus 400 include a computing device 106 such as for example the embedded computing device 106e disposed within the tote 102. The tracking apparatus 104 further includes a status module 404 that determines whether the healthcare-related items carried by the tote has been exposed to predetermined contamination risks based on environmental data captured by one or more sensors coupled to the tote. The tracking apparatus 104 also includes an action module 406 that communicates a predetermined risk mitigation action in response to determining that the tote has been exposed to the predetermined contamination risks based on one or more items of the environmental data captured being outside the predetermined range.

In certain implementations, the action module 406 predictively anticipates a risk of the conditions within the tote 102 going outside of specified ranges for the selected healthcare items. For example, if biological specimens such as organs or blood need to be maintained within a temperature range from 33° F. to 40° F. and the condition tracking apparatus 104 determines in real-time that the temperature is warming or cooling at a rate that may cause the temperature to go outside the specified range, the action module 406 may be configured to communicate an action such as to replace a lukewarm refrigerant pack with a cooler refrigerant pack or to remove condensate or spilled liquid from the tote before a specimen is impaired. Such predictive risk determination and risk mitigation action notification improves healthcare transport technology by providing actions that prevent potential risks from materializing.

In one example, the condition tracking apparatus 104 includes a location module 402 that tracks the location of the tote 102 carrying the healthcare-related items e.g., 228 by reading the identifier 214, which may include an optically scannable barcode in some examples and/or a wirelessly readable/writable RFID/NFC device 226 and other examples. In some examples, the computing device 106 reading the identifier 214 has access to the location services 114 either over the data network 110 or directly, for example, using a GPS receiver in the computing device 106. Accordingly, by determining the location of the computing device 106 reading the identifier 214, the location of the tote 102 can also be determined within a predetermined level of specificity.

In certain examples, the location module 402 stores the location of the tote 102 to a table, database, log, or other data structure in a storage device such as non-volatile memory, disk drive, cloud storage, or other suitable storage structure. In some examples, the location module 402 stores the location of the tote 102 in a non-volatile memory of the identifier 214 e.g. in a non-volatile memory of the RFID/NFC device 226. The ability to store location of the tote to device coupled to the tote itself provides a significant improvement over conventional technology by adding a level of redundancy to the tracking system which reduces the likelihood that a particular location of the tote determined at a time corresponding to specific environmental measurements could fail to be determined due to for example electromagnetic shielding at the location that prevents wireless signals from being transmitted from inside the location to external access points or due to a temporary failure of a wireless communication between the computing device 106 and the data network 110.

In one example, the apparatus 400 includes an instance of the condition tracking apparatus 104 with a status module 404 that determines whether the healthcare-related items (e.g., 228) carried by the tote 102 has been exposed to predetermined contamination risks based on environmental data captured by one or more environmental sensors e.g., 218 coupled to the tote 102. For example, as described with respect to apparatus 200 of FIG. 2A, 2B, in various examples, the sensors 218 may include temperature sensors, humidity sensors, light sensors, motion sensors, air movement sensors, or any type of sensors configurable to sense a condition related to a predetermined contamination risk around, on, or within the tote 102.

In various examples, the predetermined contamination risks may include relative humidity that falls outside a predetermined range, a temperature that falls outside of a predetermined range, exposure to air, exposure to contact, exposure to light, exposure to movement outside a predetermined range, etc. For example, some sterilization guidelines recommend that for environments surrounding or within a tote for healthcare-related items the relative humidity be maintained between 35% and 70% and the temperature be maintained between 50° F. and 72° F. and that if the temperature exceeds 120° F. the healthcare-related items be re-sterilized.

In some examples, the status module 404 queries the database that includes range parameters for predetermined contamination risks established by particular healthcare facilities, sterilization standards organizations, and the like. In certain examples, the status module 404 causes the database to be updated on a regular basis consistent with relevant healthcare guidelines.

In one example, the tracking apparatus 104 includes an action module 406 that communicates a predetermined risk mitigation action in response to the status module 404 determining that the tote (e.g., the tote 102) has been exposed to the predetermined contamination risks based on one or more items of the environmental data captured being outside the predetermined range. For example, assume that a tote 102 is carrying healthcare-related items 222 such a biological specimens that need to be maintained at a sufficiently cold temperature. In one example, in response to the status module 404 determining that a temperature sensor of the one or more sensors 218 coupled to the tote 102 detected a temperature above 40° F. within the tote 102, the action module 406 communicates a predetermined risk mitigation action to not use the healthcare-related items 222 and/or to sterilize the tote 102. In one example, the predetermined risk mitigation action is communicated to a user using a digital alert screen (e.g., one or more displays 216) forming part of the tote 102 (such as the compartment 201 and/or the sealable closure 202).

Other examples of predetermined risk mitigation actions communicated by the action module 406 includes, in various examples, sending a text or other alert signal or message to a predetermined computing device assigned to a particular station or to a particular person.

In various examples, the status module 404 maps the environmental data captured by the one or more sensors 218 to various locations tracked by the location module 402 and in response to the status module 404 determining that one or more items of the environmental data falls outside a predetermined range corresponding to a predetermined contamination risk, the action module 406 communicates the location and/or time of the contamination risk and further communicates one or more predetermined risk mitigation actions to reduce the immediate risk of contamination to the identified tote 102 and the healthcare-related items 222 carried within and/or to reduce the risk of future contamination occurring at a similar location/time.

In certain examples, the status module 404 stores data regarding a personal identifier of a user using the tote 102 and/or the computing device 106. In various examples, in response to determining that healthcare-related items e.g. 228 carried by the tote 102, has been exposed to predetermined contamination risks based on environmental data captured by the one or more sensors 218, the action module 406 communicates a predetermined risk mitigation action to the identified user of the tote 102 and/or the computing device 106 and/or to an organization or person supervising such user. Thus, the action module 406 improves sterilization technology by providing information relevant to implement improved training, procedures, and/or practices based on identifying the location, conditions, and custody of the tote 102 and the healthcare-related items 222 carried within.

FIG. 4 is a schematic block diagram of an example application of an apparatus 500 for condition tracking of a healthcare-related items tote. In one example, the apparatus 500 includes a computing device 106 with a display 502.

In various examples, the display 502 displays a name 504 associated with the identifiers 214 (including, for example, RFID/NFC devices 226) of the tote. For example, as depicted, the display 502 may display a three-character abbreviation (e.g., Intelligent Healthcare Tote abbreviated as IT) names IHT-1, IHT-2, IHT-3 for three different totes 102. In some examples, the display 502 displays a time 506 corresponding to a time that data shown on the display 502 was captured by the sensors 218.

In certain examples, the display 502 displays a location 508 corresponding to a location of the sensors 218 and/or the computing device 106 where the environmental data displayed on the display 502 was captured. In some examples, the display 502 displays a numerical representation of environmental data captured by the sensors 218 such as for example temperature 510, relative humidity 512, and/or other environmental data. In various examples, the display 502 displays processed data 514 including for example aggregated, averaged, or otherwise processed environmental data.

In some examples, the display 502 displays a graphical representation of the environmental data, e.g., a temperature scale 516 that includes an annotation showing that the temperature is rising. As another example, the display 502 in certain examples, displays a graphical representation of a relative humidity scale 518 showing that the relative humidity is following. In further examples, the graphical representation includes an alert icon 520 that indicates that the environmental data being displayed falls outside a predetermined range. The depicted examples are merely exemplary and a person of ordinary skill in the art will recognize that other configurations and other variables may be displayed by the display 502 consistent with the structures and functions of the apparatuses and methods described herein.

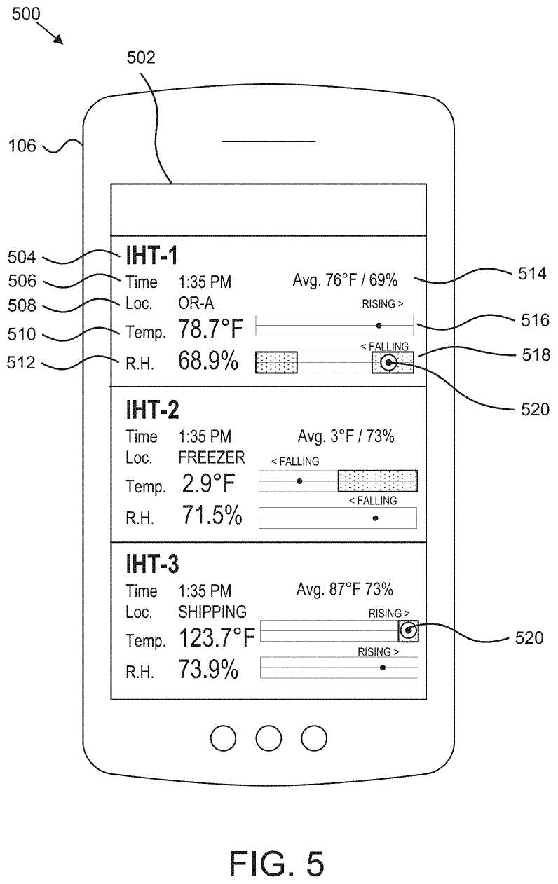

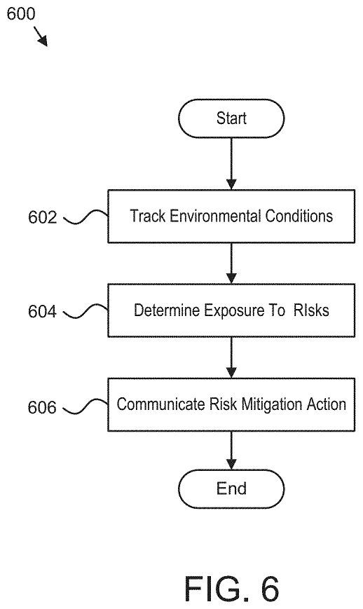

FIG. 5 is a schematic flowchart diagram of one example of a method 600 for condition tracking of a healthcare-related items tote. In one example, the method 600 begins and tracks 602 a location of the tote configured to maintain a sterilized environment for carrying healthcare-related items using an identifier coupled to the tote. In various examples, the method 600 tracks 602 a location of the tote using a location sensor such as a GPS locator or a communication device with access to location services and in data communication with the tote. In the example, the method 600 continues and determines 604 whether the healthcare-related items carried by the tote has been exposed to predetermined contamination risks based on environmental data captured by one or more sensors coupled to the tote. The method 600 continues and communicates 606 a predetermined risk mitigation action in response to determining that the tote has been exposed to the predetermined contamination risks based on one or more items of the environmental data captured being outside the predetermined range, and the method 600 ends. In various examples, the location module 402, the status module 404, and/or the action module 406 perform one or more of the steps of the method 600.

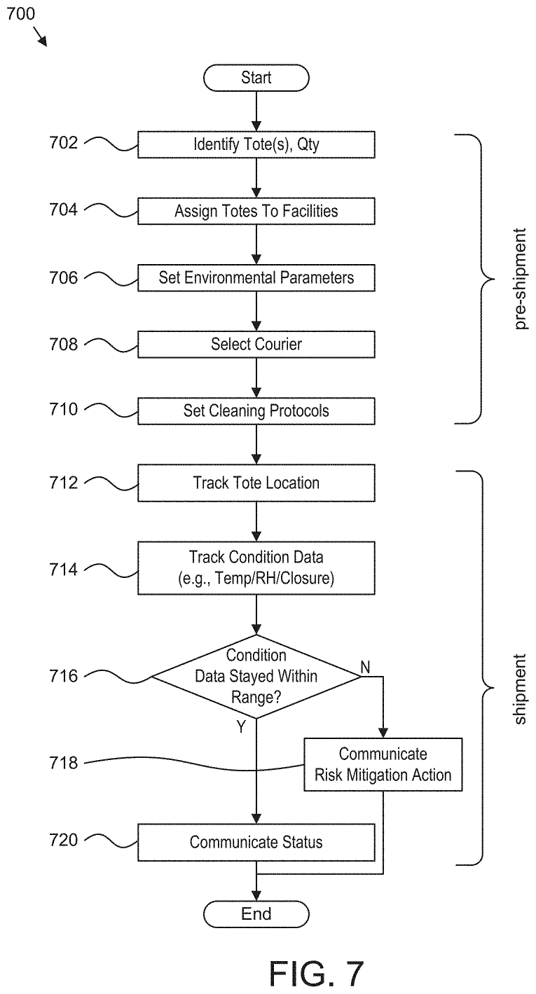

FIG. 7 is a schematic flowchart diagram of another example of a method 700 for condition tracking of a healthcare transport tote. In one example, the method 700 begins and identifies 702 a number of totes for particular facility or process. Identifying 702 the totes includes, in certain embodiments, recording or establishing the tote identifier. The tote identifier may include shared identified elements for grouping multiple totes for use within a particular facility or department within a facility.

In certain implementations, the method 700 continues and assigns 704 the one or more totes to selected facilities. For example, prior to a shipment of healthcare related items, an administrator at a healthcare facility may assign selected totes to various locations within a particular hospital, medical facility, campus, and so forth. The selection of a particular tote may be based on the type of healthcare related items to be carried within the tote and/or the mode of transportation used for transporting the tote, or other criteria as determined by the administrator.

In some implementations, the method 700 continues and sets 706 environmental parameters for the selected tote. Environmental parameters may include for example, ranges of temperature, relative humidity, closure of the tote, light, motion, air movement, duration of transport, authorized courier, or any condition related to a predetermined risk associated with the type of healthcare related items to be transported within the tote.

In various implementations, the method 700 selects 708 an authorized courier to transport the tote. For example, the method 700 may select 708 a first courier for transport of high-risk items such as radioactive materials, dangerous pathogens, delicate equipment, and so forth, and a second less specialized courier for more routine shipments not involving high-risk items. In such embodiments, selecting 708 the authorized courier may include loading contact information for the authorized courier into a system for providing text and/or email notifications.

In certain implementations, the method 700 sets 710 cleaning protocols based on factors such as the type of healthcare related items being transported, the cleaning processes available at a particular facility, cleaning recommendations or standards established by government or industry organizations, and so forth. Furthermore, specific facilities may establish cleaning protocols that exceed minimum requirements established by government or industry organizations.

In some implementations, the method 700 continues and includes tracking 714 tote locations. For example, in certain implementations, the method 700 tracks 714 the tote location by storing a location of the tote and/or the computing device reading the identifier coupled to the tote to a database stored in a computing device, a server, or in memory within an identifier coupled to the tote as described above with respect to apparatus 200 of FIGS. 2A, 2B, the apparatus 300 of FIGS. 3A, 3B, and the apparatus 400 of FIG. 4. In various examples, the method 700 continues and records 716 environmental data indicating a condition of the tote and/or compartment in which the healthcare items are transported captured by one or more sensors coupled to or disposed within a portion of the tote. For example, the sensors may sense temperature, relative humidity, closure of the tote, light, motion, air movement, or any condition related to a predetermined risk around, on, or within the tote 102.

In some examples, the method determines 716 whether the tote or the healthcare items within have been exposed to risks based on whether the environmental data remained within one or more predetermined ranges of conditions during the period of time in which the healthcare items were transported using the tote. In response to determining that the tote and/or the healthcare item have not been exposed to risks, the method 700 communicates 720 a status that the tote has remained within the predetermined range of acceptable conditions.

In response to determining that the tote has been exposed to contamination risks, the method 700 communicates 718 the one or more risk mitigation actions to mitigate the risk of contamination such as for example communicating an action to re-sterilize the healthcare-related items carried in the tote and/or to re-sterilize the tote. In some examples, the location module 402, the status module 404, the action module 406 perform one or more acts of the method 700.

In certain implementations, the method 700 includes certain acts e.g., 702, 704, 706, 708, and 710 that are performed in a pre-shipment phase i.e., prior to using the tote to transport the healthcare-related items. Similarly, in such implementation, other acts such as the acts 712, 714, 716, 718, and 720 are performed in s shipment phase, i.e., while the tote is being used to transport the healthcare related items. In various examples, one or more of the acts 702-720 of the method 700 are performed using the system 100 and/or one or more of the apparatuses 200, 300, 400, and 500 as described above and depicted respectively in FIGS. 1, 2, 3A, 3B, 4, and 5.

The present disclosure may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus comprising:
a tote comprising a compartment and a sealable closure configured to maintain a sealed environment for carrying, within the compartment, at least one healthcare-related item selected from biological specimens, healthcare-related items, and healthcare supplies;
an identifier coupled to the tote for tracking the tote; and
a sensing module comprising one or more sensors and configured to couple to the tote to capture environmental data related to the sealed environment to which the at least one healthcare item is subjected within the tote.

2. The apparatus of claim 1, further comprising a condition tracking apparatus disposed at least partially within the tote and configured to maintain a traceable record of environmental conditions within the sealed environment during transportation of the at least one healthcare item within the compartment of the tote.

3. The apparatus of claim 2, wherein the condition tracking apparatus is configured to communicate distributed ledger transactions comprising environmental data associated with the compartment of the tote during transport.

4. The apparatus of claim 1, wherein the one or more sensors are selected from time trackers, temperature sensors, liquid sensors, humidity sensors, location sensors, movement sensors, light sensors, pressure sensors, closure status sensors and combinations thereof.

5. The apparatus of claim 4, wherein the sensing module is selected from a wireless transmitter, a wireless transceiver, a built-in display, an externally coupled display, and combinations thereof.

6. The apparatus of claim 1, wherein the sensing module is configured to be removably disposed on an interior portion of the tote with the sealable closure in a closed disposition throughout transport.

7. The apparatus of claim 1, wherein the sealable closure is securely lockable in a closed disposition to prevent physical access to an interior portion of the tote.

8. The apparatus of claim 1, further comprising a sub-compartment within the tote that is configured to hold an environment regulator selected from a passive temperature regulator, an active temperature regulator, a passive humidity regulator, an active humidity regulator, and combinations thereof.

9. The apparatus of claim 1, further comprising a rechargeable power source having a charging interface configured to enable concurrent leadless charging of one or more instances of the sensing module when removed from an interior portion of one or more totes.

10. The apparatus of claim 8, wherein the charging interface comprises a wireless charging interface that inductively couples one or more instances of the sensing module to a wireless charger.

11. The apparatus of claim 1, wherein the tote is made of materials configured to substantially retain mechanical integrity and environmental sealability of the tote throughout at least one healthcare-related items cleaning processes performed on an interior and an exterior of the tote with the sealable closure in an open disposition.

12. The apparatus of claim 11, wherein the at least one healthcare-related items cleaning process is performed at temperature and time parameters that are predetermined to ensure sterilization of an interior and an exterior of the tote.

13. The apparatus of claim 1, wherein an exterior surface of the tote includes one or more stacking features that facilitate stacking of two or more instances of a component of the tote selected from the sealable closure separated from the compartment, the compartment separated from the sealable closure, and the sealable closure coupled to the compartment.

14. A condition monitoring apparatus for monitoring a condition of a tote, wherein the tote is configured to maintain a safe environment for carrying at least one healthcare-related item selected from biological specimens, healthcare-related items, healthcare supplies, and combinations thereof, the condition monitoring apparatus comprising:
a status module that determines whether the at least one healthcare-related item presents one or more predetermined risks based on environmental data captured for an interior portion of the tote; and
an action module that communicates a predetermined risk mitigation action in response to determining that at least one healthcare-related item presents the one or more predetermined risks based on one or more values of the environmental data captured being outside a predetermined range.

15. The condition monitoring apparatus of claim 14, wherein the status module and the action module are disposed on-board the tote and are configured to determine in real-time whether the predetermined risks are presented without relying on communications with devices external to the tote.

16. The condition monitoring apparatus of claim 14, further comprising a location module that tracks the tote using an identifier coupled to the tote.

17. The condition monitoring apparatus of claim 16, wherein the location module tracks the tote using a wireless communications interface that communicates with a location sensor coupled to the tote.

18. The condition monitoring apparatus of claim 14, wherein the action module communicates the predetermined risk mitigation action to a computing device over a network.

19. A method comprising:
tracking a tote comprising a compartment and a sealable closure wherein the tote is configured to maintain a sterilized environment for carrying at least one healthcare-related item selected from biological specimens, healthcare-related items, healthcare supplies, and combinations thereof using an identifier coupled to the tote;
determining whether the at least one healthcare-related item presents one or more predetermined health-related risks based on environmental data captured for an interior portion of the tote; and
communicating a predetermined risk mitigation action in response to determining that at least one healthcare-related item presents the predetermined health-related risks based on one or more values of the environmental data captured being outside a predetermined range.

20. The method of claim 19, wherein the environmental data comprises one or more parameters selected from time, temperature, humidity, location, movement, light, pressure, closure status, and combinations thereof.
